(19) United States
(12) Patent Application Publication
(10) Pub. No.: US 2016/0120523 A1
(43) Pub. Date: May 5, 2016

(54) ENCAPSULATED CRYOPROBE FOR FLEXIBLE BRONCHOSCOPE

(71) Applicant: THE JOHNS HOPKINS UNIVERSITY, Baltimore, MD (US)

(72) Inventor: Lonny B. Yarmus, Pikesville, MD (US)

(21) Appl. No.: 14/891,063

(22) PCT Filed: May 13, 2014
(86) PCT No.: PCT/US2014/037775

§ 371 Date: Nov. 13, 2015

Related U.S. Application Data
(60) Provisional application No. 61/822,526, filed on May 13, 2013.

(51) Int. Cl. A61B 10/04; A61B 1/018; A61B 10/06

(52) U.S. Cl. CPC A61B 10/04; A61B 10/06; A61B 1/018; A61B 2018/0212

(57) ABSTRACT

An embodiment in accordance with the present invention provides a device and method for capturing a tissue sample during biopsy. The device includes a sheath configured to be disposed about an outer surface of a cryoprobe. The sheath has an encapsulating device positioned at its distal end and an actuating device positioned at its proximal end. The actuating device is used to move the encapsulating device into position for capturing the tissue sample. The sheath is configured such that the sheath and cryoprobe can be removed through a surgical scope, without having to remove the scope with each biopsy sample. This device and method result in higher quality tissue samples and less trauma to patients, because the scope can be left in place throughout the duration of the procedure.

### CROSS REFERENCE TO RELATED APPLICATIONS

This application claims the benefit of U.S. Provisional Patent Application No. 61/822,526 filed on May 13, 2013, which is incorporated by reference, herein, in its entirety. U.S. Provisional Patent Application No. 61/645,801 filed on May 11, 2012, is also incorporated by reference, herein, in its entirety.

### FIELD OF THE INVENTION

The present invention relates generally to medical devices. More particularly, the present invention relates to a device for biopsy.

### BACKGROUND OF THE INVENTION

Transbronchial biopsies (TBBx) taken with standard biopsy forceps sometimes suffer from a lack of sufficient quality due to crush artifact and can be insufficient for the diagnosis of diffuse lung disease. Obtaining sufficient tissue is especially important in patients who have received lung transplants as the diagnosis of acute cellular rejection, lymphocytic bronchiolitis and infection directly impact therapy.

Recently, the use of flexible cryoprobes for TBBx has been shown to improve the diagnostic yield in the diagnosis of lung cancer when compared to standard TBBx forceps. Recent studies have shown improved samples size and architectural preservation when comparing cryoprobe to standard transbronchial forceps biopsy. A major technical difference is that standard biopsy forceps are able to be advanced through the working channel of the bronchoscope, the jaws of the forceps are opened then closed to obtain tissue and the forceps is removed through the bronchoscope which allows the bronchoscope to stay in the airway during the entire procedure. The cryoprobe can be advanced through the working channel of the bronchoscope, but after the sample is obtained by the freezing method the tissue is too large to fit through. Therefore, the working channel and thus the cryoprobe AND the bronchoscope must be removed together. The tissue is then collected off the cryoprobe, the probe is removed and then the scope is readvanced into the airway. Given the risks of bleeding during any procedure, the ability to leave the scope in the airway for the entire procedure is currently a major advantage of the forceps over the cryoprobe.

Accordingly, there is a need in the art for a device for performing biopsies of the lung without damaging the biopsied tissue and also eliminating the need to remove and reinsert the scope with each biopsy sample.

### SUMMARY OF THE INVENTION

The foregoing needs are met, to a great extent, by a device for capturing a tissue sample during biopsy using a cryoprobe including a sheath disposable about an outside surface of the cryoprobe. The sheath defines an extended lumen therethrough, such that the cryoprobe is disposable within the extended lumen. The sheath further includes a distal end and a proximal end. The device also includes an encapsulating device disposed at the distal end of the sheath. The encapsulating device is configured to capture the tissue sample. Additionally, an actuating device is configured to actuate the sheath and the encapsulating device for capture of the tissue sample.

In accordance with an aspect of the present invention, the encapsulating device further includes jaws for capturing the tissue sample. The encapsulating device is configured to prevent crushing of the tissue sample. Also, the sheath is formed integrally with the cryoprobe. The sheath has a diameter less than that of a surgical scope, such that the device can be inserted into and removed from the surgical scope during biopsy. The actuation device further includes one of a group consisting of wires or hydraulic actuation coupling an actuator, controlled by a physician, to the encapsulating device.

In accordance with another aspect of the present invention, a system for taking a biopsy tissue sample includes a cryoprobe configured to freeze the biopsy tissue sample. The system includes a sheath disposable about an outside surface of the cryoprobe. The sheath is an elongate tube defining an extended lumen therethrough, such that the cryoprobe is disposable within the extended lumen. The sheath further includes a distal end and a proximal end. The system includes an encapsulating device disposed at the distal end of the sheath, wherein the encapsulating device is configured to capture the tissue sample. Additionally, the system includes an actuating device configured to actuate the sheath and the encapsulating device for capture of the tissue sample.

In accordance with still another aspect of the present invention, the encapsulating device further includes jaws for capturing the tissue sample. The encapsulating device is configured to prevent crushing of the tissue sample. The sheath is formed integrally with the cryoprobe. The system has a diameter less than that of a surgical scope, such that the system can be inserted into and removed from the surgical scope during biopsy.

In accordance with another aspect of the present invention, a method for capturing a tissue sample during biopsy includes inserting a cryoprobe through a surgical scope and obtaining the tissue sample using the cryoprobe. The method also includes extending a sheath and an encapsulating device down a length of the cryoprobe such that the encapsulating device covers the tissue sample. Additionally, the method includes removing the cryoprobe and sheath from the surgical scope.

In accordance with still another aspect of the present invention, the method includes expanding the encapsulating device such that it can be advanced over the tissue sample. The method includes contracting the encapsulating device around the tissue sample to hold it in place. The method includes using an actuating device disposed at a proximal end of the encapsulating sheath. Additionally, the method includes using an actuating device actuated with wires to control the encapsulating device, and alternately, the method includes using an actuating device actuated with hydraulics to control the encapsulating device. The method further includes varying the pressure applied by the encapsulating device. The method also includes repeating the steps of the method in order to take multiple samples, and leaving the surgical scope in place in order to repeat the steps of the method.

### BRIEF DESCRIPTION OF THE DRAWING

The accompanying drawings provide visual representations, which will be used to more fully describe the representative embodiments disclosed herein and can be used by those skilled in the art to better understand them and their inherent advantages. In these drawings, like reference numerals identify corresponding elements and:

FIG. 1 illustrates a top down view of an encapsulating sheath and associated cryoprobe according to an embodiment of the invention.

FIG. 2 illustrates a partial sectional view of a retracted encapsulating sheath surrounding a distal tip of an associated cryoprobe.

FIG. 3 illustrates a diagram of a range of motion of an encapsulating sheath, according to an embodiment of the present invention.

FIG. 4 illustrates an encapsulating device including a net as well as the associated cryoprobe, according to an embodiment of the present invention.

FIG. 5 illustrates a flow diagram of a method of biopsy, according to an embodiment of the present invention.

### DETAILED DESCRIPTION

The presently disclosed subject matter now will be described more fully hereinafter with reference to the accompanying Drawings, in which some, but not all embodiments of the inventions are shown. Like numbers refer to like elements throughout. The presently disclosed subject matter may be embodied in many different forms and should not be construed as limited to the embodiments set forth herein; rather, these embodiments are provided so that this disclosure will satisfy applicable legal requirements. Indeed, many modifications and other embodiments of the presently disclosed subject matter set forth herein will come to mind to one skilled in the art to which the presently disclosed subject matter pertains having the benefit of the teachings presented in the foregoing descriptions and the associated Drawings. Therefore, it is to be understood that the presently disclosed subject matter is not to be limited to the specific embodiments disclosed and that modifications and other embodiments are intended to be included within the scope of the appended claims.

An embodiment in accordance with the present invention provides a device and method for capturing a tissue sample during biopsy. The device includes a sheath configured to be disposed about an outer surface of a cryoprobe. The sheath has an encapsulating device positioned at its distal end and an actuating device positioned at its proximal end. The actuating device is used to move the encapsulating device into position for capturing the tissue sample. The sheath is configured such that the sheath and cryoprobe can be removed through a surgical scope, without having to remove the scope with each biopsy sample. This device and method result in higher quality tissue samples and less trauma to patients, because the scope can be left in place throughout the duration of the procedure.

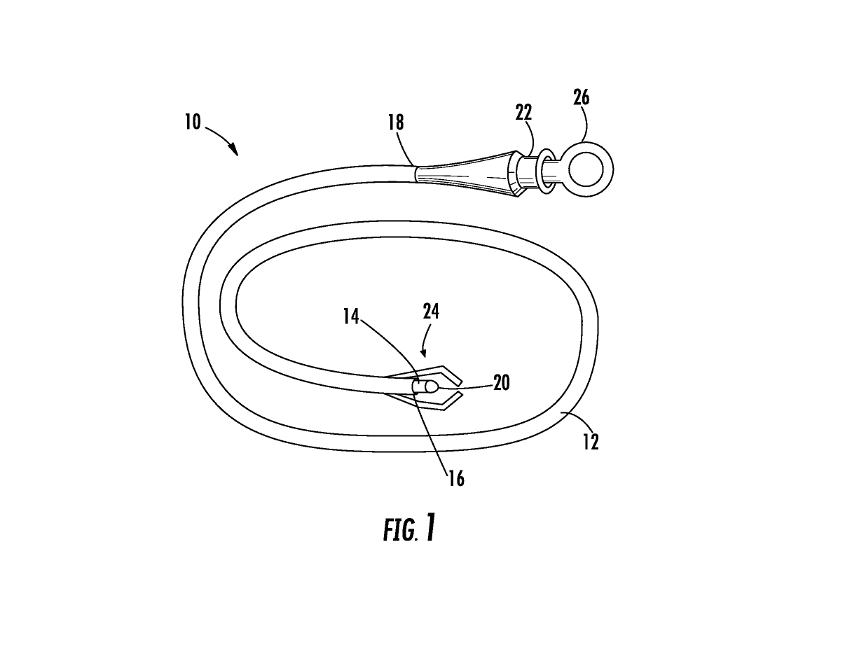

FIG. 1 illustrates a top down view of an encapsulating sheath and associated cryoprobe according to an embodiment of the invention. As illustrated in FIG. 1, a system for biopsy 10 according to the present invention can include an encapsulating sheath 12 and a cryoprobe 14. The encapsulating sheath 12 can take the form of an elongate sheath having a lumen extending therethough and having a distal end 16 and a proximal end 18. The encapsulating sheath 12 can be formed from a plastic, thermoplastic elastomer, or other biocompatible material known to or conceivable by one of skill in the art and suitable for the purpose. The cryoprobe 14 is disposable within the lumen such that the cryoprobe 14 is movable within the encapsulating sheath 12 and such that the encapsulating sheath 12 can extend from a distal end 20 of the cryoprobe 14 to a proximal end 22 of the cryoprobe 14.

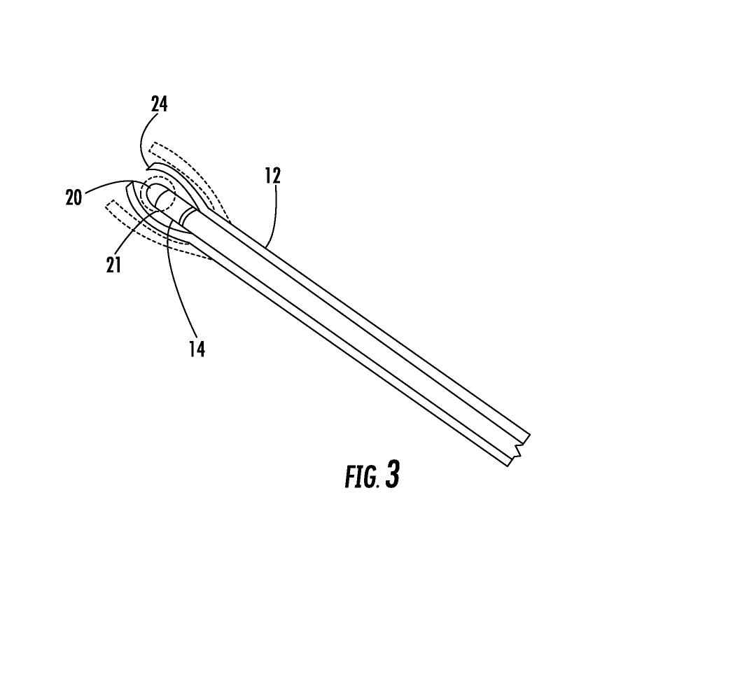

The distal end 16 of the encapsulating sheath 12 includes an encapsulating device 24 for collection of the biopsied tissue. The encapsulating device 24 can take the form of jaws that can expand and then surround a biopsy sample, as illustrated in FIG. 3. The dotted lines represent the expanded position of the encapsulating device 24 and the solid lines represent the surrounding position of the encapsulating device 24. The actuation device can help to control the pressure applied when surrounding the tissue sample when it is being surrounded by the encapsulating device. Alternately, the encapsulating device 24 can take any other suitable form known to or conceivable by one of skill in the art. The encapsulating device 24 can be formed from a metal or other biocompatible material known to or conceivable by one of skill in the art and suitable for the purpose. A diameter of the cryoprobe 14 disposed within the encapsulating sheath 12 is such that the cryoprobe 14 and encapsulating sheath 12 fit movably within a scope. Therefore, the biopsy can be taken and the cryoprobe 14, encapsulating sheath 12, and sample can be removed without also removing the scope, minimizing trauma to the patient. The proximal end 18 of the encapsulating sheath 12 includes an actuator 26 for controlling the encapsulating device 24 disposed at the distal end 16 of the encapsulating sheath 12. The actuator 26 can take any form known to or conceivable by one of skill in the art, such as wires or hydraulics coupling the actuator to the encapsulating device.

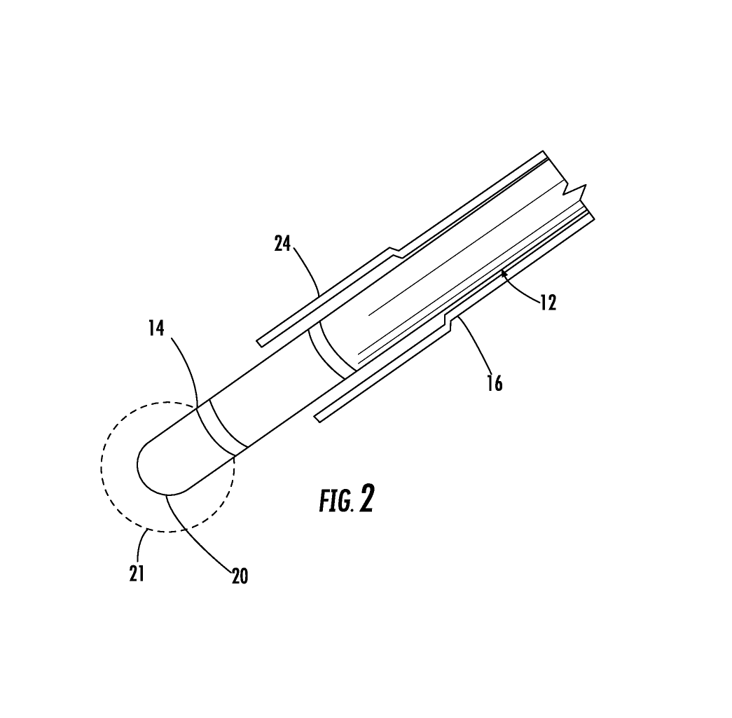

FIG. 2 illustrates a partial sectional view of a retracted encapsulating sheath surrounding a distal tip of an associated cryoprobe, and FIG. 3 illustrates a diagram of a range of motion of an encapsulating sheath, according to an embodiment of the present invention. As illustrated in FIG. 2, the encapsulating sheath 12 can be positioned away from the distal tip 20 of the cryoprobe 14, used to freeze and obtain tissue 21 for biopsy. A position of the encapsulating sheath 12 along the length of the cryoprobe 14 is controlled by the actuator 26 disposed at the proximal end of the encapsulating sheath 12. Once the tissue sample 21 is obtained the encapsulating sheath 12 and the associated encapsulating device 24 can be deployed distally to encapsulate the tissue sample for removal through the scope, as illustrated in FIG. 3. Also as illustrated in FIG. 3, the encapsulating device 24 can be configured such that it curves around the tissue sample 21 to protect it during removal. The dotted lines represent the expanded position of the encapsulating device 24 and the solid lines represent the surrounding position of the encapsulating device 24. The encapsulating device 24 can be formed integrally with the sheath or added to the sheath using adhesive, heat, or other suitable means known to one of skill in the art. The encapsulating device 24 can be formed from a shape memory metal, metal, plastic, thermoplastic elastomer, or any other suitable biocompatible material known to or conceivable by one of skill in the art.

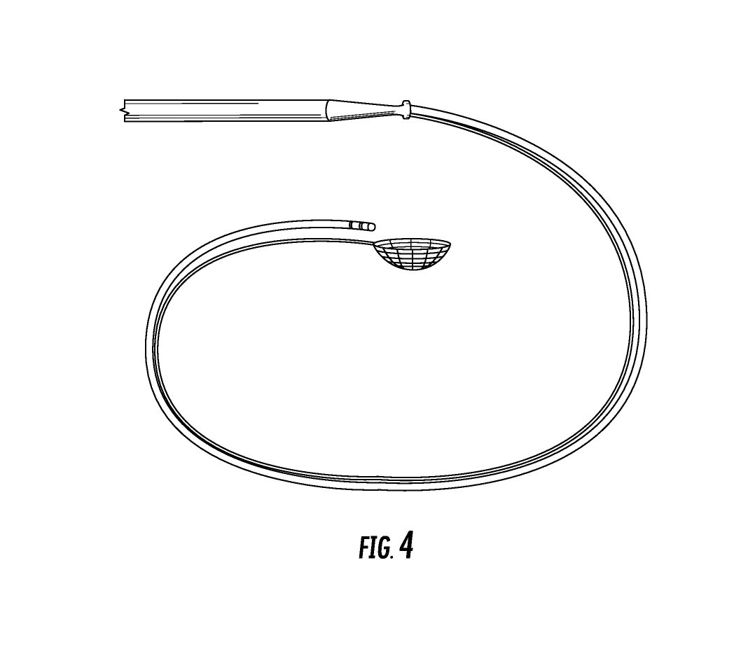

FIG. 4 illustrates an encapsulating device 50 including a net 52 as well as the associated cryoprobe 54, according to an embodiment of the present invention. As illustrated in FIG. 4, the encapsulating device 50 can also take the form of a net 52, used to capture the tissue sample after it has been frozen, obtained, and thawed off of the cryoprobe 54. The net 52 can be positioned at the distal end 56 of an encapsulating sheath 58 surrounding the cryoprobe 54 and actuated to capture the tissue sample. Alternately, the net 52 can be positioned at the distal end of a guide wire 60 and advanced adjacent or along the side of the cryoprobe, to capture the tissue sample. The net 52 can be formed from a metal, polyester fiber, nylon fiber, or other suitable biocompatible and flexible material. The guide wire 60 can be formed from a shape memory metal, such as NiTiNol. The guide wire 60 can be helically wound, straight, or have any other configuration known to or conceivable by one of skill in the art.

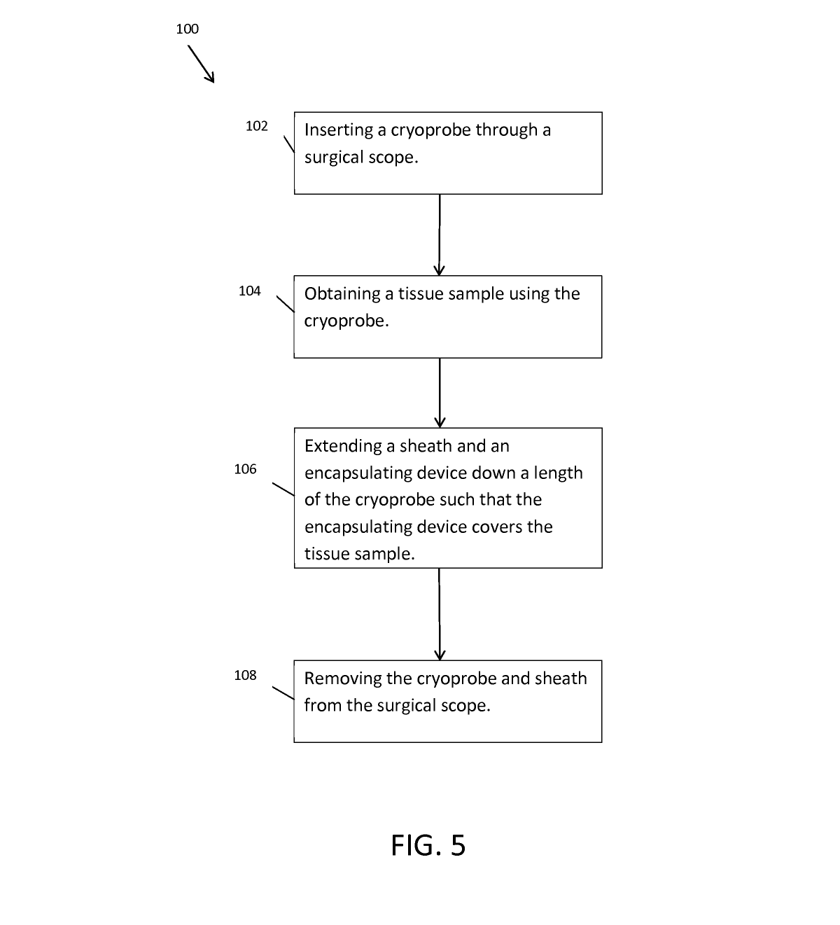

FIG. 5 illustrates a flow diagram of a method of biopsy, according to an embodiment of the present invention. The method 100 includes a step 102 of inserting a cryoprobe through a surgical scope. Step 104 includes obtaining the tissue sample using the cryoprobe. Step 106 includes extending a sheath and an encapsulating device down a length of the cryoprobe such that the encapsulating device covers the tissue sample, and step 108 includes removing the cryoprobe and sheath from the surgical scope.

While the present invention is discussed as a separate encapsulating sheath component for use with a cryoprobe, the present invention could also be integrated directly into the cryoprobe. The encapsulating sheath can be for one-time-use or reuasable and configured to be autoclaved between uses. The many features and advantages of the invention are apparent from the detailed specification, and thus, it is intended by the appended claims to cover all such features and advantages of the invention which fall within the true spirit and scope of the invention. Further, since numerous modifications and variations will readily occur to those skilled in the art, it is not desired to limit the invention to the exact construction and operation illustrated and described, and accordingly, all suitable modifications and equivalents may be resorted to, falling within the scope of the invention.

1. A device for capturing a tissue sample during biopsy using a cryoprobe comprising:
a sheath disposable about an outside surface of the cryoprobe, wherein the sheath is an elongate tube defining an extended lumen therethrough, such that the cryoprobe is disposable within the extended lumen, and wherein the sheath further includes a distal end and a proximal end;
an encapsulating device disposed at the distal end of the sheath, wherein the encapsulating device is configured to capture the tissue sample; and
an actuating device configured to actuate the sheath and the encapsulating device for capture of the tissue sample.

2. The device of claim 1 wherein the encapsulating device further comprises jaws for capturing the tissue sample.

3. The device of claim 1 wherein the encapsulating device is configured to prevent crushing of the tissue sample.

4. The device of claim 1 wherein the sheath is formed integrally with the cryoprobe.

5. The device of claim 1 further comprising a diameter less than that of a surgical scope, such that the device can be inserted into and removed from the surgical scope during biopsy.

6. The device of claim 1 wherein the actuation device further comprises one of a group consisting of wires or hydraulic actuation coupling an actuator, controlled by a physician, to the encapsulating device.

7. A system for taking a biopsy tissue sample comprising:
a cryoprobe configured to freeze the biopsy tissue sample;
a sheath disposable about an outside surface of the cryoprobe, wherein the sheath is an elongate tube defining an extended lumen therethrough, such that the cryoprobe is disposable within the extended lumen, and wherein the sheath further includes a distal end and a proximal end;
an encapsulating device disposed at the distal end of the sheath, wherein the encapsulating device is configured to capture the tissue sample;
an actuating device configured to actuate the sheath and the encapsulating device for capture of the tissue sample.

8. The system of claim 7 wherein the encapsulating device further comprises jaws for capturing the tissue sample.

9. The system of claim 7 wherein the encapsulating device is configured to prevent crushing of the tissue sample.

10. The system of claim 7 wherein the sheath is formed integrally with the cryoprobe.

11. The system of claim 7 further comprising a diameter less than that of a surgical scope, such that the device can be inserted into and removed from the surgical scope during biopsy.

12. A method for capturing a tissue sample during biopsy comprising:
inserting a cryoprobe through a surgical scope;
obtaining the tissue sample using the cryoprobe;
extending a sheath and an encapsulating device down a length of the cryoprobe such that the encapsulating device covers the tissue sample; and
removing the cryoprobe and sheath from the surgical scope.

13. The method of claim 12 further comprising expanding the encapsulating device such that it can be advanced over the tissue sample.

14. The method of claim 12 further comprising contracting the encapsulating device around the tissue sample to hold it in place.

15. The method of claim 12 further comprising using an actuating device disposed at a proximal end of the encapsulating sheath.

16. The method of claim 15 further comprising using an actuating device actuated with wires to control the encapsulating device.

17. The method of claim 15 further comprising using an actuating device actuated with hydraulics to control the encapsulating device.

18. The method of claim 15 further comprising varying the pressure applied by the encapsulating device.

19. The method of claim 11 further comprising repeating the steps of the method in order to take multiple samples.

20. The method of claim 11 further comprising leaving the surgical scope in place in order to repeat the steps of the method.
